(19) United States
(12) Patent Application Publication
(10) Pub. No.: US 2012/0020923 A1
(43) Pub. Date: Jan. 26, 2012

(54) Method of treating viral diseases with combinations of TPO receptor agonist and anti-viral agents

(76) Inventors: Connie L. Erickson-Miller, Collegeville, PA (US); Julian Jenkins, Collegeville, PA (US); Dickens Theodore, Durham, NC (US)

(21) Appl. No.: 13/190,730

(22) Filed: Jul. 26, 2011

Related U.S. Application Data
(63) Continuation of application No. 12/518,336, filed on Jun. 9, 2009, now abandoned.
(60) Provisional application No. 60/869,583, filed on Dec. 12, 2006.

(51) Int. Cl. A61K 38/21; A61P 31/14; A61K 31/7056

(52) U.S. Cl. 424/85.7; 514/43

(57) ABSTRACT

Invented is a method of treating viral diseases, particularly hepatitis C, in a human, in need thereof which comprises the administration of a combination of therapeutically active agents selected from a TPO receptor agonist and an antiviral therapy selected from: an alpha interferon, ribavirin, a ribavirin analog and an HCV antiviral to such human.

This application is a Continuation of U.S. application Ser. No. 12/518,336, filed Jun. 9, 2009, which is a 371 of PCT/US07/86918 filed Dec. 10, 2007 which claims the benefit of U.S. Provisional App. No. 60/869,583 filed Dec. 12, 2006.

### FIELD OF THE INVENTION

This invention relates to novel combinations of thrombopoietin (TPO) receptor agonists, suitably non-peptide TPO receptor agonist, and antiviral therapy selected from: alpha interferons, including Pegasys® (peginterferon alfa-2a) and Peg-Intron® (peginterferon alfa-2b), ribavirin, ribavirin analogs and HCV therapy, including HCV polymerase inhibitors, helicase inhibitors and protease inhibitors, and optional further active agents, and the use of these combinations in the treatment of viral diseases, particularly hepatitis C.

### BACKGROUND OF THE INVENTION

Thrombopoietin (TPO) has been shown to be the main humoral regulator in situations involving thrombocytopenia. See, e.g., Metcalf Nature 369:519-520 (1994). TPO has been shown in several studies to increase platelet counts, increase platelet size, and increase isotope incorporation into platelets of recipient animals. Because platelets (thrombocytes) are necessary for blood clotting and when their numbers are very low a patient is at risk of death from catastrophic hemorrhage. TPO is considered to have potential useful applications in both the diagnosis and the treatment of various hematological disorders, for example, diseases primarily due to platelet defects. In addition, studies have provided a basis for the projection of efficacy of TPO therapy in the treatment of thrombocytopenia, and particularly thrombocytopenia resulting from chemotherapy, radiation therapy, or bone marrow transplantation as treatment for cancer or lymphoma. See e.g., McDonald (1992) Am. J. Ped. Hematology/Oncology 14: 8-21 (1992).

The slow recovery of platelet levels in patients suffering from thrombocytopenia is a serious problem, and has lead to the search for TPO receptor agonists, suitably small molecule non-peptide TPO receptor agonists, that are able to accelerate platelet regeneration. (e.g. see, International Application Number PCT/US01/16863, having International Filing Date May 24, 2001).

Thrombocytopenia is defined as a reduction in platelet count; and the clinical consequences or symptoms may include petechiae, bruising, and mucosal bleeding. Ultimately, severe thrombocytopenia may be associated with morbidity and mortality due to bleeding. Thrombocytopenia is a frequent finding in several medical disorders, including aplastic anemia, myelodysplasia, and idiopathic thrombocytopenic purpura (ITP). Thrombocytopenia is also a frequently observed in subjects with advanced chronic liver disease. [Streiff, M. B. et al. Hepatology. 2002:35(4):947-952].

In hepatitis C subjects, thrombocytopenia may be attributable to hypersplenism resulting from portal hypertension, the auto-immune pathology of the hepatitis C virus and/or the reduced production of thrombopoietin by the liver [Medina, J. et al. Aliment Pharmacol Ther. 2002:20:129-141; Peck-Radosavljevic, Met al. Journal of Hepatology. 1997:27:127-131; Dieterich, D and Spivak, J. L. CID. 2003:37:533-541]. Progressive, advanced liver fibrosis leads to increased resistance to portal venous inflow within the liver, resulting in portal hypertension which causes hyperslpenism and increased sequestration of platelets. The concentration of endogenous thrombopoietin is lower in cirrhotic subjects than in normal control subjects, because the liver is the main organ responsible for thrombopoietin production [Kaushansky, K. New England Journal of Medicine, 1998:339(11):746-754]. There are several auto-immune manifestations of chronic hepatitis C virus infection including cryoglobulinemia, glomerulonephritis and the rare presence of platelet-associated immunoglobulins [Nagamine, T. et al., Journal of Hepatology. 1996:24:135-140].

One of the most common etiologies of thrombocytopenia in hepatitis C subjects is the myelosuppressive effects of the anti-viral agents interferon-alpha. Pegylated interferon-alpha is associated with neutropenia and thrombocytopenia in hepatitis C subjects. A higher incidence of thrombocytopenia is observed with pegylated interferon-alpha than non-pegylated forms, due to the higher weekly dose administered and the prolonged exposure of the bone marrow. [Dieterich, D and Spivak, J. L. CID. 2003:37:533-541; Rajan, Set al., American Journal of Hematology. 2001:68:202-209; Wang, Q. et al., Blood, 2002:96(6):2093-2099; Sagir, A et al., Digestive Diseases and Sciences, 2002:47(3):562-563]. In pivotal phase III studies with pegylated interferon 2a (Pegasys®, Roche) and 2b (Peg-Intron®, Schering-Plough) approximately 50% of subjects had a reduction in platelet count due to the antiviral therapy. Furthermore, 3-6% of subjects had their pegylated interferon dose reduced or discontinued due to thrombocytopenia. In addition, the use of pegylated interferon alpha in subjects with hepatitis C is contraindicated in subjects with pre-treatment thrombocytopenia. [Pegasys and Peg-Intron prescribing information].

Current clinical management of thrombocytopenia in patients with chronic hepatitis C relies primarily on the reduction of pegylated interferon alpha dose. A 50% dose reduction of Pegasys and Peg-Intron is recommended when platelet counts are <50,000 and <80,000 platelets/uL respectively and discontinuation when <25,000 and <50,000 platelets/uL respectively. However, reducing the pegylated interferon dose by ≧20% is associated with reduced antiviral efficacy [McHutchinson, J. G. et al., Gastroenterology, 2002:123(4)1061-1069]. Therefore, there is an unmet need for a safe and effective treatment of disease- and interferon-associated thrombocytopenia in hepatitis C subjects.

It would be desirable to provide combinations that provide enhanced treatment of hepatitis C.

The present invention relates to therapeutic uses of a novel combination of known classes of compounds, TPO receptor agonists, suitably non-peptide TPO receptor agonists, and antiviral therapy selected from: alpha interferons, including Pegasys® (peginterferon alfa-2a) and Peg-Intron® (peginterferon alfa-2b), ribavirin, ribavirin analogs and HCV therapy, including HCV polymerase inhibitors, helicase inhibitors and protease inhibitors, and optional further active agents. The present invention concerns a method for treating hepatitis C by administering such combination to a subject in need thereof.

As disclosed herein it has been discovered that a combination of a TPO receptor agonist, suitably a non-peptide TPO receptor agonist, and an antiviral therapy selected from: alpha interferons, ribavirin, ribavirin analogs and HCV therapy, and optional further active agents are useful in treating hepatitis C.

As disclosed herein it has been discovered that the in vivo administration of a TPO receptor agonist, suitably a non-peptide TPO receptor agonist, and an antiviral therapy selected from: alpha interferons, ribavirin, ribavirin analogs and HCV therapy, is useful in treating hepatitis C.

### SUMMARY OF THE INVENTION

This invention relates to novel combinations of thrombopoietin (TPO) receptor agonists, suitably non-peptide TPO receptor agonists, and antiviral therapy selected from: alpha interferons, including Pegasys® (peginterferon alfa-2a) and Peg-Intron® (peginterferon alfa-2b), ribavirin, ribavirin analogs and HCV therapy, including HCV polymerase inhibitors, helicase inhibitors and protease inhibitors, and optional further active agents.

This invention relates to a method of treating viral diseases, particularly hepatitis C, in a human in need thereof which comprises administering to such subject a therapeutically effective amount of a TPO receptor agonist, suitably a non-peptide TPO receptor agonist, and an antiviral therapy selected from: alpha interferons, ribavirin, ribavirin analogs and HCV therapy.

This invention also relates to the discovery that the in vivo administration of a TPO receptor agonist, suitably a non-peptide TPO receptor agonist, and an antiviral therapy selected from: alpha interferons, ribavirin, ribavirin analogs and HCV therapy provides enhanced therapeutic treatment of viral diseases, particularly hepatitis C.

Included among the TPO receptor agonist of the invention is AMG 531.

Included among the non-peptide TPO receptor agonists of the invention are compounds of Formula (I):

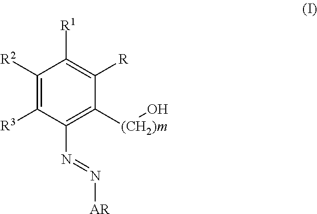

wherein:
R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid, —SO₂NR⁵R⁶, and a heterocyclic methylene substituent as represented by Formula (III),

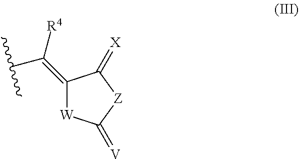

where,
p is 0-6,
n is 0-2,
V, W, X and Z are each independently selected from O, S and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R⁴ is selected from: hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
m is 0-6; and
AR is a cyclic or polycyclic aromatic ring containing from 3 to 16 carbon atoms and containing one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR⁴, —C(O)NR¹⁰R¹¹, —S(O)₂NR¹⁰R¹¹, —S(O)ₙR⁴ and protected —OH,
where n is 0-2,
R⁴ is hydrogen, alkyl, cycloalkyl, C₁-C₁₂aryl, substituted alkyl, substituted cycloalkyl and substituted C₁-C₁₂aryl, and
R¹⁰ and R¹¹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR⁴, —S(O)ₙR⁴, —C(O)NR⁴R⁴, —S(O)₂NR⁴R⁴, nitro, cyano, cycloalkyl, substituted cycloalkyl, halogen, aryl, substituted aryl and protected —OH, or R¹⁰ and R¹¹ taken together with the nitrogen to which they are attached represent a 5 to 6 member saturated ring containing up to one other heteroatom selected from oxygen and nitrogen,
where R⁴ is as described above and n is 0-2;
and/or pharmaceutically acceptable salts thereof;
provided that at least one of R, R¹, R² and R³ is a substituted aryl group or a heterocyclic methylene substituent as represented in Formula (III).

This invention relates to a method of treating viral diseases, particularly hepatitis C, which comprises administering to a subject in need thereof a therapeutically effective amount of a TPO receptor agonist, suitably a non-peptide TPO receptor agonist of Formula (I) and an antiviral therapy selected from: alpha interferons, ribavirin, ribavirin analogs and HCV therapy.

Included in the present invention are pharmaceutical combinations and methods of use thereof that comprise a TPO receptor agonist, suitably a non-peptide TPO receptor agonist, and an antiviral therapy selected from: alpha interferons, ribavirin, ribavirin analogs and HCV therapy, and optionally a further active agent and a pharmaceutical carrier.

### DETAILED DESCRIPTION OF THE INVENTION

This invention relates to methods of treating viral diseases, particularly hepatitis C, in a human in need thereof which comprises in vivo administration to such human a combination of therapeutically active agents selected from: a TPO receptor agonist, suitably a non-peptide TPO receptor agonist, including compounds of Formula (I) as described above and antiviral therapy selected from: alpha interferons, including Pegasys® (peginterferon alfa-2a) and Peg-Intron® (peginterferon alfa-2b), ribavirin, ribavirin analogs and HCV therapy, including HCV polymerase inhibitors, helicase inhibitors and protease inhibitors, and optional further active agents.

Included among the non-peptide TPO receptor agonists that are useful in the present invention are those having Formula (V):

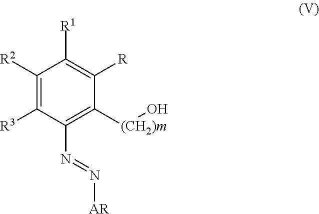

wherein:
R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid and —SO₂NR⁵R⁶,
where,
p is 0-6,
n is 0-2,
R⁴ is selected from: hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
m is 0-6; and
AR is a cyclic or polycyclic aromatic ring containing from 3 to 16 carbon atoms and one or more heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, aryl, substituted cycloalkyl, substituted aryl, aryloxy, oxo, hydroxy, alkoxy, cycloalkyl, acyloxy, amino, N-acylamino, nitro, cyano, halogen, —C(O)OR⁴, —C(O)NR¹⁰R¹¹, —S(O)₂NR¹⁰R¹¹, —S(O)ₙR⁴ protected —OH,
where n is 0-2,
R⁴ is hydrogen, alkyl, cycloalkyl, C₁-C₁₂aryl, substituted alkyl, substituted cycloalkyl and substituted C₁-C₁₂aryl; and
R¹⁰ and R¹¹ are independently hydrogen, cycloalkyl, C₁-C₁₂aryl, substituted cycloalkyl, substituted C₁-C₁₂aryl, alkyl or alkyl substituted with one or more substituents selected from the group consisting of: alkoxy, acyloxy, aryloxy, amino, N-acylamino, oxo, hydroxy, —C(O)OR⁴, —S(O)ₙR⁴, —C(O)NR⁴R⁴, —S(O)₂NR⁴R⁴, nitro, cyano, cycloalkyl, substituted cycloalkyl, halogen, aryl, substituted aryl and protected —OH, or R¹⁰ and R¹¹ taken together with the nitrogen to which they are attached represent a 5 to 6 member saturated ring containing up to one other heteroatom selected from oxygen and nitrogen,
where R⁴ is as described above and n is 0-2;
and/or a pharmaceutically acceptable salt thereof;
provided that at least one of R, R¹, R² and R³ is a substituted aryl group.

Included among the compounds that are useful in the present invention are those having Formula (II):

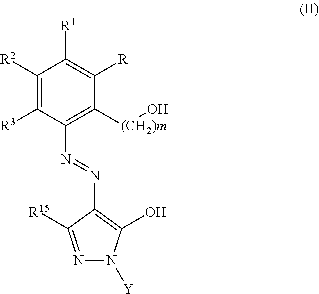

wherein:
R, R¹, R² and R³ are each independently selected from hydrogen, C₁₋₆alkyl, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid, —SO₂NR⁵R⁶, and a heterocyclic methylene substituent as represented by Formula (III),

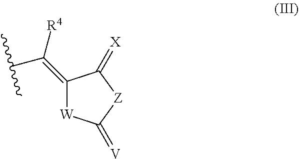

where
p is 0-6,
n is 0-2,
V, W, X and Z are each independently selected from O, S, and NR¹⁶, where R¹⁶ is selected from: hydrogen, alkyl, cycloalkyl, C₁-C₁₂aryl, substituted alkyl, substituted cycloalkyl and substituted C₁-C₁₂aryl,
R⁴ is hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
R¹⁵ is selected from the group consisting of alkyl, C₁-C₁₂aryl, hydroxy, alkoxy, substituted alkyl, substituted C₁-C₁₂aryl and halogen;
m is 0-6; and
Y is selected from alkyl, substituted alkyl and a cyclic or polycyclic aromatic ring containing from 3 to 14 carbon atoms and optionally containing from one to three heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH;
and/or pharmaceutically acceptable salts thereof;
provided that at least one of R, R¹, R² and R³ is a substituted aryl group or a heterocyclic methylene substituent as represented in Formula (III).

Included among compounds of Formula (II) that are useful in the current invention are those having Formula (VI):

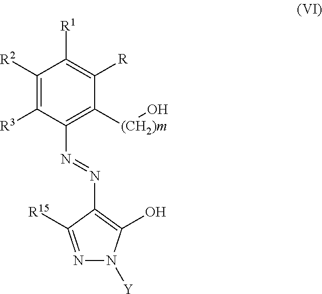

wherein:
R, R¹, R² and R³ are each independently selected from hydrogen, C₁₋₆alkyl, C₁₋₆alkoxy, —(CH₂)ₚOR⁴, —C(O)OR⁴, formyl, nitro, cyano, halogen, aryl, substituted aryl, substituted alkyl, —S(O)ₙR⁴, cycloalkyl, —NR⁵R⁶, protected —OH, —CONR⁵R⁶, phosphonic acid, sulfonic acid, phosphinic acid and —SO₂NR⁵R⁶,
where
p is 0-6,
n is 0-2,
R⁴ is hydrogen, alkyl, cycloalkyl, C₁-C₁₂aryl, substituted alkyl, substituted cycloalkyl and substituted C₁-C₁₂aryl, and
R⁵ and R⁶ are each independently selected from hydrogen, alkyl, substituted alkyl, C₃₋₆cycloalkyl, and aryl,
or R⁵ and R⁶ taken together with the nitrogen to which they are attached represent a 5 to 6 member saturated ring containing up to one other heteroatom selected from oxygen and nitrogen;
R¹⁵ is selected from the group consisting of alkyl, C₁-C₁₂aryl, hydroxy, alkoxy, substituted alkyl, substituted C₁-C₁₂aryl and halogen;
m is 0-6; and
Y is selected from alkyl, substituted alkyl and a cyclic or polycyclic aromatic ring containing from 3 to 14 carbon atoms and optionally containing from one to three heteroatoms, provided that when the number of carbon atoms is 3 the aromatic ring contains at least two heteroatoms and when the number of carbon atoms is 4 the aromatic ring contains at least one heteroatom, and optionally substituted with one or more substituents selected from the group consisting of: alkyl, substituted alkyl, C₁-C₁₂aryl, substituted cycloalkyl, substituted C₁-C₁₂aryl, hydroxy, aryloxy, alkoxy, cycloalkyl, nitro, cyano, halogen and protected —OH;
and/or pharmaceutically acceptable salts thereof;
provided that at least one of R, R¹, R² and R³ is a substituted aryl group.

Included among the compounds useful in the present invention are those having Formula (VI) in which,

either:
R is a substituted aryl; and R¹ is hydrogen;
   or:
R is hydrogen; and R¹ is a substituted aryl;
   and in either case:
R² and R³ are each independently selected from hydrogen, C₁₋₆alkyl, C₁₋₆alkoxy, nitro, cyano, halogen, aryl, substituted aryl, substituted alkyl, cycloalkyl, phosphonic acid, phosphinic acid and sulfonic acid;
R¹⁵ is selected from the group consisting of alkyl, substituted alkyl, C₁-C₁₂aryl, alkoxy and halogen;
m is 0-4; and
Y is selected from,
phenyl, pyridinyl and pyrimidinyl, where the phenyl, pyridinyl and pyrimidinyl are optionally substituted with from one to three substituents selected from the group consisting of: alkyl, substituted alkyl, C₁-C₁₂aryl, substituted C₁-C₁₂aryl, alkoxy and halogen;
and/or pharmaceutically acceptable salts thereof.

Included among the compounds useful in the present invention are those having Formula (VI) in which,
R is a substituted C₁-C₁₂aryl;
and
R¹ is hydrogen;
R² and R³ are each independently selected from hydrogen, C₁₋₆alkyl, C₁₋₆alkoxy, nitro, cyano, halogen, substituted alkyl and cycloalkyl;
R¹⁵ is selected from the group consisting of alkyl, substituted alkyl, C₁-C₁₂aryl, alkoxy and halogen;
m is 0-2; and
Y is selected from,
phenyl, pyridinyl and pyrimidinyl, where the phenyl, pyridinyl and pyrimidinyl are optionally substituted with from one to three substituents selected from the group consisting of: alkyl, substituted alkyl, C₁-C₁₂aryl, substituted C₁-C₁₂aryl, alkoxy and halogen;
and/or pharmaceutically acceptable salts thereof.

Included among the compounds useful in the present invention are those having Formula (VI) in which,
R is a substituted phenyl or pyridinyl ring; and
R¹ is hydrogen;
R² and R³ are each independently selected from hydrogen, C₁₋₆alkyl, substituted alkyl and halogen;
R¹⁵ is selected from the group consisting of C₁₋₄alkyl, C₁₋₄alkoxy, C₁-C₁₂ aryl and halogen;
m is 0; and
Y is selected from,
phenyl, pyridinyl and pyrimidinyl, where the phenyl, pyridinyl and pyrimidinyl is optionally substituted with from one to three substituents selected from the group consisting of: alkyl, substituted alkyl, C₁-C₁₂aryl, substituted C₁-C₁₂aryl, alkoxy and halogen;
and/or pharmaceutically acceptable salts thereof.

Included among the compounds useful in the present invention are:
4′-{N′-[1-(3,4-Dimethylphenyl)-3-methyl-5-oxo-1,5-dihydropyrazol-4-ylidene]hydrazino}-3′-hydroxybiphenyl-4-carboxylic acid;
4′-{N′-[1-(3,4-Dimethylphenyl)-3-methyl-5-oxo-1,5-dihydropyrazol-4-ylidene]hydrazino}-3′-hydroxybiphenyl-3-carboxylic acid;
3′-{N′-[1-(3,4-Dimethylphenyl)-3-methyl-5-oxo-1,5-dihydropyrazol-4-ylidene]hydrazino}-2′-hydroxybiphenyl-3-carboxylic acid;
3′-{N′-[1-(4-tert-Butylphenyl)-3-methyl-5-oxo-1,5-dihydropyrazol-4-ylidene]hydrazino}-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-5′-chloro-2′-hydroxybiphenyl-3-carboxylic acid;
2-Aza-3′-{N′-1-(4-tert-butylphenyl)-3-methyl-5-oxo-1,5-dihydropyrazol-4-ylidene]hydrazino}-2′-hydroxybiphenyl-3-carboxylic acid;
3-Aza-3′-{N′-[1-(4-tert-butylphenyl)-3-methyl-5-oxo-1,5-dihydropyrazol-4-ylidene]hydrazino}-2′-hydroxybiphenyl-5-carboxylic acid;
2-Aza-5′-chloro-3′-{N′-[1-(3,4-dimethylphenyl)-3-methyl-5-oxo-1,5-dihydropyrazol-4-ylidene]hydrazino}-2′-hydroxybiphenyl-3-carboxylic acid;
2-Aza-3′-{N′-[1-(4-tert-butylphenyl)-3-methyl-5-oxo-1,5-dihydropyrazol-4-ylidene]hydrazino}-2′-hydroxy-5′-methylbiphenyl-3-carboxylic acid;
2-Aza-3′-{N′-[1-(3,4-dimethylphenyl)-3-methyl-5-oxo-1,5-dihydropyrazol-4-ylidene]hydrazino}-2′-hydroxy-5′-methylbiphenyl-3-carboxylic acid;
3′-{N′-[1-(4-tert-Butylphenyl)-3-methyl-5-oxo-1,5-dihydropyrazol-4-ylidene]hydrazino}-2′-hydroxy-5′-methylbiphenyl-3-carboxylic acid;
3-{N′-[1-(3,4-dimethylphenyl)-3-methyl-5-oxo-1,5-dihydropyrazol-4-ylidene]hydrazino}-2-hydroxy-3′-(tetrazol-5-yl)biphenyl;
3′-{N′-[1-(3,4-dimethylphenyl)-3-methyl-5-oxo-1,5-dihydropyrazol-4-ylidene]hydrazino}-5′-fluoro-2′-hydroxybiphenyl-3-carboxylic acid;
7-({N′-[1-(3,4-dimethylphenyl)-3-methyl-5-oxo-1,5-dihydropyrazol-4-ylidene]hydrazino}-2-hydroxyphenyl)quinolin-4[1H]-one-3-carboxylic acid;
7-({N′-[1-(4-tert-butylphenyl)-3-methyl-5-oxo-1,5-dihydropyrazol-4-ylidene]hydrazino}-2-hydroxyphenyl)quinolin-4[1H]-one-3-carboxylic acid;
3-Aza-3′-{N′-[1-(3,4-dimethylphenyl)-3-methyl-5-oxo-1,5-dihydropyrazol-4-ylidene]hydrazino}-2′-hydroxybiphenyl-5-carboxylic acid;
3-Aza-3′-(N′-[1-{3-methyl-[4-(1-methylethyl)phenyl]-5-oxo-1,5-dihydropyrazol-4-ylidene}hydrazino)-2′-hydroxybiphenyl-5-carboxylic acid;
3-Aza-3′-{N′-[1-(4-tertbutylphenyl-3-methyl-5-oxo-1,5-dihydropyrazol-4-ylidene]hydrazino}-2′-hydroxybiphenyl-5-carboxylic acid;
5′-Chloro-3′-{N′-[1-(3,4-dimethylphenyl)-3-methyl-5-oxo-1,5-dihydropyrazol-4-ylidene]hydrazino}-2′-hydroxybiphenyl-3-carboxylic acid;
3′-{N′-[1-(3,4-Dimethylphenyl)-3,5-dioxo-1,5-dihydropyrazol-4-ylidene]hydrazino}-2′-hydroxybiphenyl-3-carboxylic acid;
3′-{N′-[1-(2-Ethoxy-2-oxoethyl)-3-methyl-5-oxo-1,5-dihydropyrazol-4-ylidene]hydrazino}-2′-hydroxybiphenyl-3-carboxylic acid;
3-{N′-[1-(3,4-dimethylphenyl)-3-methyl-5-oxo-1,5-dihydropyrazol-4-ylidene]hydrazino}-2-hydroxy-4′-(tetrazol-5-yl)biphenyl;
3′-(N′-{1-[2-(N-tert-butyl)amino-2-oxoethyl]-3-methyl-5-oxo-1,5-dihydropyrazol-4-ylidene}hydrazino)-2′-hydroxybiphenyl-3-carboxylic acid;
3′-{N′-[3-Chloro-1-(3,4-dimethylphenyl)-5-oxo-1,5-dihydropyrazol-4-ylidene]hydrazino}-2′-hydroxybiphenyl-3-carboxylic acid;
5-chloro-3-{N′-[1-(3,4-dimethylphenyl)-3-methyl-5-oxo-1,5-dihydropyrazol-4-ylidene]hydrazino}-2-hydroxy-4′-(tetrazol-5-yl)biphenyl;
3′-{N′-[1-(3,4-Dimethylphenyl)-3-methyl-5-oxo-1,5-dihydropyrazol-4-ylidene]hydrazino}-2′-hydroxybiphenyl-3,5-dicarboxylic acid;
3-Aza-3′-{N′-[1-(3,4-dimethylphenyl)-3-methyl-5-oxo-1,5-dihydropyrazol-4-ylidene]hydrazino}-2′-hydroxy-5′-methylbiphenyl-5-carboxylic acid;
3′-{N′[1-(3,4-Dimethylphenyl)-3-methyl-5-oxo-1,5-dihydropyrazol-4-ylidene]hydrazino}-2′-hydroxybiphenyl-4-carboxylic acid;
3′-{N′-[1-(3,4-Dimethylphenyl)-3-methoxy-5-oxo-1,5-dihydropyrazol-4-ylidene]hydrazino}-2′-hydroxybiphenyl-3-carboxylic acid;
3′-{N′-[1-(4-methoxyphenyl)-3-methyl-5-oxo-1,5-dihydropyrazol-4-ylidene]hydrazino}-2′-hydroxybiphenyl-3-carboxylic acid;
(3-{N′-[1-(3,4-dimethylphenyl)-3-methyl-5-oxo-1,5-dihydropyrazol-4-ylidene]hydrazino}-2-hydroxy-3′-biphenyl)-1,1,1,-trifluoromethanesulfonamide;
3′-{N′-[1-(3,4-Dichlorophenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(3-trifluoromethylphenyl)-1,5-dihydropyrazol-4-ylidene]hydrazino}-2′-hydroxybiphenyl-3-carboxylic acid;
8-{N′-[1-(3,4-dimethylphenyl)-3-methyl-5-oxo-1,5-dihydropyrazol-4-ylidene]hydrazino}quinolin-4[1H]-one-3-carboxylic acid;
3′-{N′-[3-methyl-5-oxo-1-(4-trifluoromethylphenyl)-1,5-dihydropyrazol-4-ylidene]hydrazino}-2′-hydroxybiphenyl-3-carboxylic acid;
3′-{N′-[3-methyl-5-oxo-1-(4-N-methylcarboxamidolphenyl)-1,5-dihydropyrazol-4-ylidene]hydrazino}-2′-hydroxybiphenyl-3-carboxylic acid;
N-[1-(3′-{N′-[1-(3,4-dimethylphenyl)-3-methyl-5-oxo-1,5-dihydropyrazol-4-ylidene]hydrazino}-2′-hydroxybiphenyl-3-yl)methanoyl]methanesulfonamide;
3′-{N′-[3-methyl-5-oxo-1-phenyl-1,5-dihydropyrazol-4-ylidene]hydrazino}-2′-hydroxybiphenyl-3-carboxylic acid;
3′-{N′-[3-methyl-1-(4-methylphenyl)-5-oxo-1,5-dihydropyrazol-4-ylidene]hydrazino}-2′-hydroxybiphenyl-3-carboxylic acid;
3′-{N′-[1-(4-chlorophenyl)-3-methyl-5-oxo-1,5-dihydropyrazol-4-ylidene]hydrazino}-2′-hydroxybiphenyl-3-carboxylic acid;
3′-{N′-[1-(4-fluorophenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(4-trifluoromethoxyphenyl)-1,5-dihydropyrazol-4-ylidene]hydrazino}-2′-hydroxybiphenyl-3-carboxylic acid;
3′-{N′-[1-(3,4-dimethylphenyl)-3-ethoxy-5-oxo-1,5-dihydropyrazol-4-ylidene]hydrazino}-2′-hydroxybiphenyl-3-carboxylic acid;
3′-{N′-[1-(3,4-dimethylphenyl)-3-(1-methylethoxy)-5-oxo-1,5-dihydropyrazol-4-ylidene]hydrazino}-2′-hydroxybiphenyl-3-carboxylic acid;
3′-{N′-[3-tert-butyl-1-(3,4-dimethylphenyl)-5-oxo-1,5-dihydropyrazol-4-ylidene]hydrazino}-2′-hydroxybiphenyl-3-carboxylic acid;
3′-{N′3-methyl-1-(4-methyl-2,3,5,6-tetrafluorophenyl)-5-oxo-1,5-dihydropyrazol-4-ylidene]hydrazino}-2′-hydroxybiphenyl-3-carboxylic acid;
3′-{N′-[1-(4-fluoro-3-methylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-phenyl-5-oxo-1,5-dihydropyrazol-4-ylidene]hydrazino}-2′-hydroxybiphenyl-3-carboxylic acid;
3-{N′-[1-(3,4-dimethylphenyl)-5-oxo-3-phenyl-1,5-dihydropyrazol-4-ylidene]hydrazino}-2-hydroxy-3′-tetrazol-5-ylbiphenyl;
3-{N′-[1-(3,4-dimethylphenyl)-3-methoxy-5-oxo-1,5-dihydropyrazol-4-ylidene]hydrazino}-2-hydroxy-3′-tetrazol-5-ylbiphenyl;
3-{N′-[1-(3,4-dimethylphenyl)-3-ethoxy-5-oxo-1,5-dihydropyrazol-4-ylidene]hydrazino}-2-hydroxy-3′-tetrazol-5-ylbiphenyl;
3-{N′-[1-(3,4-dimethylphenyl)-3-(1-methylethoxy)-5-oxo-1,5-dihydropyrazol-4-ylidene]hydrazino}-2-hydroxy-3′-tetrazol-5-ylbiphenyl;
3-{N′-[1-(4-fluorophenyl)-3-methyl-5-oxo-1,5-dihydropyrazol-4-ylidene]hydrazino}-2-hydroxy-3′-tetrazol-5-ylbiphenyl;
3-{N′-[1-(4-fluoro-3-methylphenyl)-3-methyl-5-oxo-1,5-dihydropyrazol-4-ylidene]hydrazino}-2-hydroxy-3′-tetrazol-5-ylbiphenyl;
3-{N′-[3-methyl-5-oxo-1-(4-trifluoromethylphenyl)-1,5-dihydropyrazol-4-ylidene]hydrazino}-2-hydroxy-3′-tetrazol-5-ylbiphenyl;
3′-{N′-[1-(3,4-dimethylphenyl)-3-(pyridin-4-yl-5-oxo-1,5-dihydropyrazol-4-ylidene]hydrazino}-2′-hydroxybiphenyl-3-carboxylic acid;
3-{N′-[1-(3,4-dimethylphenyl)-3-pyridin-4-yl-5-oxo-1,5-dihydropyrazol-4-ylidene]hydrazino}-2-hydroxy-3′-tetrazol-5-ylbiphenyl;
3-{N′-[1-(3,4-dimethylphenyl)-3-pyridin-2-yl-5-oxo-1,5-dihydropyrazol-4-ylidene]hydrazino}-2-hydroxy-3′-tetrazol-5-ylbiphenyl;
3′-{N′-[1-(3,4-dimethylphenyl)-3-(pyridin-2-yl-5-oxo-1,5-dihydropyrazol-4-ylidene]hydrazino}-2′-hydroxybiphenyl-3-carboxylic acid;
3-{N′-[1-(3-fluoro-4methylphenyl)-3-methyl-5-oxo-1,5-dihydropyrazol-4-ylidene]hydrazino}-2-hydroxy-3′-tetrazol-5-ylbiphenyl;
3′-{N′-[1-(3-fluoro-4-methylphenyl)-3-methyl-5-oxo-1,5-dihydropyrazol-4-ylidene]hydrazino}-2′-hydroxybiphenyl-3-carboxylic acid;
3′-{N′-[3-methyl-5-oxo-1-(4-trifluoromethylpyrimidin-2-yl)-1,5-dihydropyrazol-4-ylidene]hydrazino}-2′-hydroxybiphenyl-3-carboxylic acid;
3′-N-tert-butoxycarbonylamino-3-{N′-[1-(3,4-Dimethylphenyl)-3-methyl-5-oxo-1,5-dihydropyrazol-4-ylidene]hydrazino}-2-hydroxybiphenyl;
3′-amino-3-{N′-[1-(3,4-dimethylphenyl)-3-methyl-5-oxo-1,5-dihydropyrazol-4-ylidene]hydrazino}-2-hydroxybiphenyl;
3-{N′-[1-(3-fluorophenyl)-3-methyl-5-oxo-1,5-dihydropyrazol-4-ylidene]hydrazino}-2-hydroxy-3′-tetrazol-5-ylbiphenyl;
3′-{N′-[1-(3-fluorophenyl)-3-methyl-5-oxo-1,5-dihydropyrazol-4-ylidene]hydrazino}-2′-hydroxybiphenyl-3-carboxylic acid;
3-{N′-[3-methyl-5-oxo-1-(2,3,4,5,6-pentafluorophenyl)-1,5-dihydropyrazol-4-ylidene]hydrazino}-2-hydroxy-3′-tetrazol-5-ylbiphenyl;
3′-{N′-[3-methyl-5-oxo-1-(2,3,4,5,6-pentafluorophenyl)-1,5-dihydropyrazol-4-ylidene]hydrazino}-2′-hydroxybiphenyl-3-carboxylic acid;
3′-{N′-[1-(3,4-difluorophenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methoxymethyl-5-oxo-1,5-dihydropyrazol-4-ylidene]hydrazino}-2′-hydroxybiphenyl-3-carboxylic acid;
3-{N′-[1-(3,4-dimethylphenyl)-3-methoxymethyl-5-oxo-1,5-dihydropyrazol-4-ylidene]hydrazino}-2-hydroxy-3′-tetrazol-5-ylbiphenyl;
3-{N′-[1-(3,4-difluorophenyl)-3-methyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-5-oxo-3-trifluoromethyl-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-6-fluoro-2′-hydroxybiphenyl-3-carboxylic acid;
3′-{N′-[1-(3,4-dimethylphenyl)-5-oxo-3-propyl-1,5-dihydropyrazol-4-ylidene]hydrazino}-2′-hydroxybiphenyl-3-carboxylic acid;
3-{N′-[1-(3,4-dimethylphenyl)-5-oxo-3-propyl-1,5-dihydropyrazol-4-ylidene]hydrazino}-2-hydroxy-3′-tetrazol-5-ylbiphenyl;
3′-{N′-[1-(3,4-dimethylphenyl)-3-(1-methyl-1H-pyrrol-3-yl)-5-oxo-1,5-dihydropyrolzol-4-ylidene]hydrazino}-2′-hydroxybiphenyl-3-carboxylic acid;
3-{N′-[1-(3,4-dimethylphenyl)-3-(1-methyl-1H-pyrrol-3-yl)-5-oxo-1,5-dihydropyrazol-4-ylidene]hydrazino}-2-hydroxy-3′-tetrazol-5-ylbiphenyl;
3′-{N′-[1-(3,4-dimethylphenyl)-3-furan-2-yl-5-oxo-1,5-dihydropyrazol-4-ylidene]hydrazino}-2′-hydroxybiphenyl-3-carboxylic acid;
3-{N′-[1-(3,4-dimethylphenyl)-3-furan-2-yl-5-oxo-1,5-dihydropyrazol-4-ylidene]hydrazino}-2-hydroxy-3′-tetrazol-5-ylbiphenyl;
N-(2′-hydroxy-3′-{N′-[3-methyl-5-oxo-1-(4-trifluoromethyl-phenyl)-1,5-dihydropyrazol-4-ylidene]hydrazino}biphenyl-3-yl)-1,1,1-trifluoromethanesulfonamide;
N-(2′-hydroxy-3′-{N′-[1-(3-fluoro-4-methylphenyl)-3-methyl-5-oxo-1,5-dihydropyrazol-4-ylidene]hydrazino}biphenyl-3-yl)-1,1,1-trifluoromethanesulfonamide;
N-(2′-hydroxy-3′-{N′-[1-(4-fluoro-3-methylphenyl)-3-methyl-5-oxo-1,5-dihydropyrazol-4-ylidene]hydrazino}biphenyl-3-yl)-1,1,1-trifluoromethanesulfonamide;
N-(2′-hydroxy-3′-{N′-[1-(3,4-difluorophenyl)-3-methyl-5-oxo-1,5-dihydropyrazol-ylidene]hydrazino}biphenyl-3-yl)-1,1,1-trifluoromethanesulfonamide;
N-(3′-{N′-[1-(3,4-dimethylphenyl)-3-methyl-5-oxo-1,5-dihydropyrazol-4-ylidene]hydrazino}-2′-hydroxybiphenyl-3-yl)guanidine;
3′-{N′-[1-(3,4-dimethylphenyl)-3-ethyl-5-oxo-1,5-dihydropyrazol-4-ylidene]hydrazino}-2′-hydroxybiphenyl-3-carboxylic acid;
3-{N′-[1-(3,4-dimethylphenyl)-3-ethyl-5-oxo-1,5-dihydropyrazol-4-ylidene]hydrazino}-2-hydroxy-3′-tetrazol-5-ylbiphenyl;
3′-{N′-[1-(3,4-dimethylphenyl)-5-oxo-3-thien-2-yl-1,5-dihydropyrazol-4-ylidene]hydrazino}-2′-hydroxybiphenyl-3-carboxylic acid;
3′-{N′-[3-cyclopropyl-1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-5-oxo-3-thiazol-2-yl-1,5-dihydropyrazol-4-ylidene]hydrazino}-2′-hydroxybiphenyl-3-carboxylic acid;
3′-{N′-[1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-3-(1-methylethyl)-5-oxo-1,5-dihydropyrazol-4-ylidene]hydrazino}-2′-hydroxybiphenyl-3-carboxylic acid;
3′-{N′-[3-(benzyloxymethyl)-1-(3,4-dimethylphenyl)-5-oxo-1,5-dihydropyrazol-4-ylidene]hydrazino}-2′-hydroxybiphenyl-3-carboxylic acid;
3′-{N′-[3-ethyl-5-oxo-1-(4-trifluoromethylphenyl)-1,5-dihydropyrazol-4-ylidene]hydrazino}-2′-hydroxybiphenyl-3-carboxylic acid;
3′-{N′-[5-oxo-1-(4-trifluoromethylphenyl)-1,5-dihydropyrazol-4-ylidene]hydrazino}-2′-hydroxybiphenyl-3-carboxylic acid;
3′-{N′-[-1-(3,4-dimethylphenyl)-3-hydroxymethyl-5-oxo-1,5-dihydropyrazol-4-ylidene]hydrazino}-2′-hydroxybiphenyl-3-carboxylic acid;
3′-{N′-[3-benzyloxymethyl-5-oxo-1-(4-trifluoromethylphenyl)-1,5-dihydropyrazol-4-ylidene]hydrazino}-2′-hydroxybiphenyl-3-carboxylic acid;
3′-{N′-[-1-(3,4-dimethylphenyl)-3-methylsulfanylmethyl-5-oxo-1,5-dihydropyrazol-4-ylidene]hydrazino}-2′-hydroxybiphenyl-3-carboxylic acid;
3′-{N′-[-1-(3,4-dimethylphenyl)-5-oxo-3-thiophen-3-yl-1,5-dihydropyrazol-4-ylidene]hydrazino}-2′-hydroxybiphenyl-3-carboxylic acid;
3′-{N′-[5-oxo-1-(4-trifluoromethylphenyl)-3-thiophen-3-yl-1,5-dihydropyrazol-4-ylidene]hydrazino}-2′-hydroxybiphenyl-3-carboxylic acid;
3′-{N′-[5-oxo-1-(4-trifluoromethylphenyl)-3-methylsulfanylmethyl-1,5-dihydropyrazol-4-ylidene]hydrazino}-2′-hydroxybiphenyl-3-carboxylic acid;
N-(3′-{N′-[1 -(3,4-dimethylphenyl)-3-methyl-5-oxo-1,5-dihydropyrazol-4-ylidene]hydrazino}-2′-hydroxybiphenyl-3-yl)methanesulfonamide;
3′-[N′-(1-benzo[1,3]dioxol-5-yl-3-methyl-5-oxo-1,5-dihydropyrazol-4-ylidene)hydrazino]-2′-hydroxybiphenyl-3-carboxylic acid;
3′-{N′-[1-(3,5-dimethylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-4′-hydroxybiphenyl-4-carboxylic acid;
3′-{N′-[1-(3-chloro-4-methylphenyl)-3-methyl-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-4′-hydroxybiphenyl-3-carboxylic acid;
3′-{N′-[1-(3,4-dimethylphenyl)-3-methyl-5-oxo-1,5-dihydropyrazol-4-ylidene]hydrazino}-2′-hydroxybiphenyl-3-phosphonic acid;
3′-{N′-[1-(3,4-dimethylphenyl)-3-methyl-5-oxo-1,5-dihydropyrazol-4-ylidene]hydrazino}-2′-hydroxybiphenyl-3,4-dicarboxylic acid;
2′,6-dihydroxy-3′-{N′-[1-(3,4-dimethylphenyl)-3-methyl-5-oxo-1,5-dihydropyrazol-4-ylidene]hydrazino}biphenyl-3-carboxylic acid;
4-aza-3′-{N′-[1-(3,4-dimethylphenyl)-3-methyl-5-oxo-1,5-dihydropyrazol-4-ylidene]hydrazino}-2′-hydroxybiphenyl-5-carboxylic acid;
3′-{N′-[1-(3,4-dimethylphenyl)-5-oxo-1,5-dihydropyrazol-4-ylidene]hydrazino}-2′-hydroxybiphenyl-3-carboxylic acid;
3′-{N′-[1-(3,4-dimethylphenyl)-3-methyl-5-oxo-1,5-dihydropyrazol-4-ylidene]hydrazino}-2′-hydroxybiphenyl-3-sulfonic acid; and
5-(3′-{N′-[1-(3,4-Dimethylphenyl)-3-methyl-5-oxo-1,5-dihydropyrazol-4-ylidene]hydrazino}-2′-hydroxybiphenyl-3-ylmethylene)thiazolidine-2,4-dione;
and/or pharmaceutically acceptable salts thereof.

Included among the non-peptide TPO receptor agonists of the invention are the non-peptide compounds described in:
WO 02/59099;
WO 02/59100;
EP 1 207 155;
EP 1 253 142A1;
WO 01/92211 A1;
WO 01/53267-A1;
EP 1 104 674-A1;
WO 01/07423-A1;
WO 05/118551; and
WO 06/047344.

Included among the compounds of the above listed applications that are useful in the present invention are:

N-[4-(5-bromo-2-thienyl)-1,3-thiazol-2-yl]-4-[(Z)-(2,4-dioxo-1,3-thiazolidin-5-ylidene)methyl]benzamide;

N-[4-(3,4-dimethylphenyl)-1,3-thiazol-2-yl]-4-[(Z)-(2,4-dioxo-1,3-thiazolidin-5-ylidene)methyl]benzamide;

N-{4-[4-(1,1-dimethylethyl)phenyl]-1,3-thiazol-2-yl}-4-[(Z)-(2,4-dioxo-1,3-thiazolidin-5-ylidene)methypenzamide;

N-[4-(3,4-dichlorophenyl)-1,3-thiazol-2-yl]-4-[(Z)-(2,4-dioxo-1,3-thiazolidin-5-ylidene)methyl]benzamide; and

(2E)-3-[4-({[4-(3,4-dichlorophenyl)-1,3-thiazol-2-yl]amino}carbonyl)phenyl]-2-methyl-2-propenoic acid;

and/or pharmaceutically acceptable salts, hydrates, solvates and esters thereof.

Included among the non-peptide TPO receptor agonists of the invention are the non-peptide compounds described in:
WO 99/11262.

Included among the TPO receptor agonists of the invention is the well known protein AMG 531.

Included among the non-peptide TPO receptor agonists of the invention are the non-peptide compounds described in:

International Application No. PCT/US05/018924, having an International filing date of May 27, 2005; International Publication Number WO 05/118551 and an International Publication date of Dec. 15, 2005,

International Application No. PCT/US05/038055, having an International filing date of Oct. 21, 2005; International Publication Number WO 06/047344 and an International Publication date of May 4, 2006,

International Application No. PCT/US06/045129, having an International filing date of Nov. 21, 2006; International Publication Number WO 07/062078 and an International Publication date of May 31, 2007, and

International Application No. PCT/US07/006547, having an International filing date of Mar. 14, 2007; International Publication Number WO 07/106564 and an International Publication date of Sep. 20, 2007, suitably the compound of Example 4 therein: 3′-{N′-[1-(3,5-Dimethyl-phenyl)-2-oxo-6-trifluoromethyl-1,2-dihydro-indol-3-ylidene]hydrazino}-2′-hydroxybiphenyl-3-carboxylic acid, or a pharmaceutically acceptable salt thereof;

which is a compound having the structure:

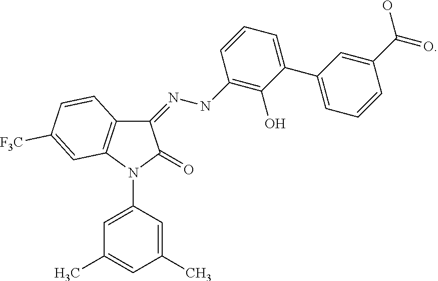

The compounds that are final products in both WO 05/118551, WO 06/047344, WO 07/062078 and WO 07/106564 are useful in the present invention, these compounds are included herein by reference.

Included among the non-peptide TPO receptor agonists of the invention is the non-peptide compound described in:

International Application No. PCT/JP03/012419, having an International filing date of Sep. 29, 2003; International Publication Number WO 04/029049 and an International Publication date of Apr. 8, 2004, 2005.

The compound that is the final product in WO 04/029049, both the salt and non-salt forms, is useful in the present invention, these compounds are included herein by reference.

Suitably, the compound that is the final product in WO 04/029049 is 1-(3-chloro-5-{[4-(4-chlorothiophen-2-yl)-5-(4-cyclohexylpiperazin-1-yl)thiazol-2-yl]carbamoyl}pyridine-2-yl)piperidine-4-carboxylic acid, as the salt free compound or in the form of a maleic acid salt. The structure of the free or unsalted compound is indicated below.

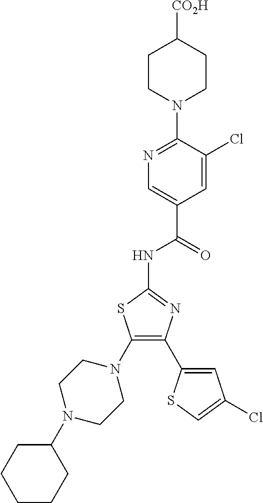

Non-peptide TPO receptor agonists, and pharmaceutical compositions thereof, are included in the combinations of the invention and used in the methods of the invention.

Included among the alpha interferons of the invention are the well known interferons: Pegasys® (peginterferon alfa-2a) and Peg-Intron® (peginterferon alfa-2b).

By the term ribavirin analogs as used herein are chemical compounds which maintain the same -tetrahydrofuran-2-yl-1,2,4-triazole-core structure as ribavirin but which compounds contain chemical modifications to one or more of the substituents attached to the core structure or at one or more of the reactive sites around the core structure. Suitably, the ribavirin analog will contain from 1 to 3, suitably 1 or 2, suitably 1 chemical modification to a substituent attached to the core structure. Suitably, the ribavirin analog will contain from 1 to 3, suitably 1 or 2, suitably 1 chemical modification to a reactive site around the core structure.

By the term “protected hydroxy” or “protected —OH” as used herein, is meant the alcoholic or carboxylic-OH groups which can be protected by conventional blocking groups in the art such as described in “Protective Groups In Organic Synthesis” by Theodora W. Greene, Wiley-Interscience, 1981, New York. Compounds containing protected hydroxy groups may also be useful as intermediates in the preparation of the pharmaceutically active compounds of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, furan, pyrrole, pyrazole, imidazole and tetrazole.

When referring to compounds of Formula (I) and (II), the term “substituted” as used herein, unless otherwise defined, is meant that the subject chemical moiety has one or more substituents selected from the group consisting of: —CO₂R²⁰, aryl, —C(O)NHS(O)₂R²⁰, —NHS(O)₂R²⁰, hydroxyalkyl, alkoxy, —C(O)NR²¹R²², acyloxy, alkyl, amino, N-acylamino, hydroxy, —(CH₂)_{g}C(O)OR⁸, —S(O)ₙR⁸, nitro, tetrazole, cyano, oxo, halogen, trifluoromethyl, protected —OH and a heterocyclic methylene substituent as represented by Formula (III),

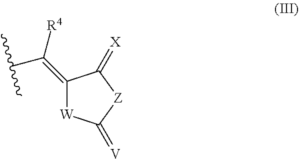

, where g is 0-6; R⁸ is hydrogen or alkyl; R²⁰ is selected form hydrogen, C₁-C₄alkyl, aryl and trifluoromethyl; R²¹ and R²² are independently selected form hydrogen, C₁-C₄alkyl, aryl and trifluoromethyl; V, W, X and Z are each independently selected from O, S, and NR¹⁶, where R¹⁶ is selected from: hydrogen, alkyl, cycloalkyl, C₁-C₁₂aryl, substituted alkyl, substituted cycloalkyl and substituted C₁-C₁₂aryl; and n is 0-2.

When referring to compounds of Formula (V) and (VI), the term “substituted” as used herein, unless otherwise defined, is meant that the subject chemical moiety has one or more substituents selected from the group consisting of: —CO₂R²⁰, aryl, —C(O)NHS(O)₂R²⁰, —NHS(O)₂R²⁰, R²⁰, hydroxyalkyl, alkoxy, —C(O)NR²¹R²², acyloxy, alkyl, amino, N-acylamino, hydroxy, —(CH₂)_{g}C(O)OR⁸, —S(O)ₙR⁸, nitro, tetrazole, cyano, oxo, halogen, trifluoromethyl and protected —OH, where g is 0-6, R⁸ is hydrogen or alkyl, R²⁰ is selected form hydrogen, C₁-C₄alkyl, aryl and trifluoromethyl, and R²¹ and R²² are independently selected form hydrogen, C₁-C₄alkyl, aryl and trifluoromethyl, and n is 0-2.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C_{12.}

Examples of cycloalkyl and substituted cycloalkyl substituents as used herein include: cyclohexyl, 4-hydroxy-cyclohexyl, 2-ethylcyclohexyl, propyl 4-methoxycyclohexyl, 4-methoxycyclohexyl, 4-carboxycyclohexyl, cyclopropyl and cyclopentyl.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifuloromethyl, acyloxy, amino, N-acylamino, hydroxy, —(CH₂)_{g}C(O)OR⁸, —S(O)ₙR⁸, nitro, cyano, halogen and protected —OH, where g is 0-6, R⁸ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein is meant a linear or branched, saturated or unsaturated hydrocarbon chain, and unless otherwise defined, the carbon chain will contain from 1 to 12 carbon atoms. Examples of alkyl substituents as used herein include: —CH₃, —CH₂—CH₃, —CH₂—CH₂—CH₃, —CH(CH₃)₂, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylactic and therapeutic therapy.

By the phrases “to a therapeutic extent” and “therapeutically effective amount” and derivatives thereof as used herein, unless otherwise defined, is meant that the treatment of a viral disease, particularly hepatitis C, in patients treated with a combination of an antiviral therapy selected from: alpha interferons, ribavirin, ribavirin analogs and HCV therapy, and a TPO receptor agonist, suitably a non-peptide TPO receptor agonist, and optional further active agent or agents, is enhanced in comparison to patients being treated with an antiviral therapy selected from: alpha interferons, ribavirin, ribavirin analogs and HCV therapy, alone or such antiviral therapy and optional further active agent or agents in the absence of a TPO receptor agonist, suitably a non-peptide TPO receptor agonist. By the term “enhanced” as used herein, unless otherwise defined, is meant that such antiviral therapy, in the presence or absence of a further active agent or agents, is administered for a longer period of time and/or in larger amounts, resulting in a greater antiviral activity, when administered to patients in combination with a TPO receptor agonist, suitably a non-peptide TPO receptor agonist, then when such antiviral therapy is administered in the absence of a TPO receptor agonist, suitably a non-peptide TPO receptor agonist. The TPO receptor agonist, suitably non-peptide TPO receptor agonist, provides an increase in the platelet count enabling an increased and/or extended treatment with such antiviral therapy. Additionally, treatment with a TPO receptor agonist, suitably non-peptide TPO receptor agonist, and the resultant increase in platelet count, provides treatment with an antiviral therapy selected from: alpha interferons, ribavirin, ribavirin analogs and HCV therapy, in the presence or absence of a further active agent or agents, to a therapeutic extent in that such antiviral therapy could not be administered in any amount in the absence of the TPO receptor agonist, suitably non-peptide TPO receptor agonist, and the resultant increase in platelet count.

By the phrase “non-peptide” as used herein is meant a chemical compound, or a protein or peptide not comprised primarily of natural amino acids. Suitably, the “non-peptide” is a small molecule chemical compound having a molecular weight under 1,500 daltons, suitably under 1,000 daltons, suitably under 750 daltons.

By the term “primarily” as used above is meant about 60% by weight of naturally occurring amino acid residue, suitably 80%.

Compounds of Formula (I) are included in the combinations of the invention and used in the methods of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

The compounds of Formulas I and II are disclosed and claimed, along with pharmaceutically acceptable salts, hydrates, solvates and esters thereof, as being useful as an agonist of the TPO receptor, particularly in enhancing platelet production and particularly in the treatment of thrombocytopenia, in International Application No. PCT/US01/16863, having an International filing date of May 24, 2001; International Publication Number WO 01/89457 and an International Publication date of Nov. 29, 2001, the entire disclosure of which is hereby incorporated by reference. Compounds of Formulas I and II and pharmaceutically acceptable salts, hydrates, solvates and esters thereof, are prepared as described in International Application No. PCT/US01/16863. The bis-(monoethanolamine) salt of a compound described in International Application No. PCT/US01/16863, is described in International Application No. PCT/US03/16255, having an International filing date of May 21, 2003; International Publication Number WO 03/098992 and an International Publication date of December 4, 2003.

By the term “combination” and derivatives thereof as used herein is meant either simultaneous administration or any manner of separate sequential administration of the subject therapeutically active agents. Preferably, if the administration is not simultaneous, the compounds are administered in a close time proximity to each other. Furthermore, it does not matter if the compounds are administered in the same dosage form, e.g. one compound may be administered topically and another compound may be administered orally. Regarding the present invention, a “combination” requires, at a minimum, one TPO receptor agonist, suitably a non-peptide TPO receptor agonist, as described herein, and an antiviral therapy selected from: alpha interferons, ribavirin, ribavirin analogs and HCV therapy. Also contemplated for use herein is the optional co-administration of a further active agent or agents with the “combination”.

By the term “co-administration” and derivatives thereof as used herein is meant either simultaneous administration or any manner of separate sequential administration of therapeutically active agents of a combination of the invention, including optional further active agents. Preferably, if the administration is not simultaneous, the compounds are administered in a close time proximity to each other. Furthermore, it does not matter if the compounds are administered in the same dosage form, e.g. one compound may be administered topically and another compound may be administered orally.

By the term “therapeutically active agents” and derivatives thereof as used herein is meant TPO receptor agonist, suitably non-peptide TPO receptor agonist, and antiviral therapy selected from: alpha interferons, ribavirin, ribavirin analogs and HCV therapy and optional further active agents.

Examples of further active agents for use with the presently invented combinations means compounds known to treat viral diseases or assist in the treatment of viral diseases, particularly hepatitis C or compounds known or found to be useful when administered to a subject in need of treatment for viral diseases, particularly hepatitis C. Examples of further active agents for use herein include but are not limited to the well known compound ribavirin.

Prophylactic use of the combinations of this invention is contemplated whenever possible exposure to a viral disease, particularly hepatitis C, is anticipated, or after a known exposure to a viral disease, particularly hepatitis C, but before a diagnosis of viral infection.

One skilled in the art can readily determine by known methods if a compound is a TPO receptor agonist, suitably a non-peptide TPO receptor agonist, and thus included within the scope of the current invention. By way of example, the following assays can be employed:

### Luciferase Assay

Compounds are tested for potency as agonists of the TPO receptor in a Luciferase assay such as described in Lamb, et al., Nucleic Acids Research 23: 3283-3289 (1995) and Seidel, et al., Proc. Natl. Acad. Sci., USA 92: 3041-3045 (1995) by substituting a TPO-responsive BaF3 cell line (Vigon et al. Proc. Natl. Acad. Sci. USA 1992, 89, 5640-5644) for the HepG2 cells utilized therein. The murine BaF3 cells express TPO receptors and closely match the pattern of STAT (signal transducers and activators of transcription) activation observed in primary murine and human bone marrow cells.

### Proliferation Assay

Compounds are tested in an in vitro proliferation assay using the human UT7TPO cell line. UT7TPO cells are a human megakaryoblastic cell line that express Tpo-R, whose survival and growth is dependent on the presence of TPO (Komatsu et al. Blood 1996, 87,4552).

### Differentiation Assay

Compounds are tested for their ability in stimulating the maturation of megakaryocytes from human bone marrow cells. In this assay, purified human CD34+ progenitor cells are incubated in liquid culture with test compounds for 10 days and the number of cells expressing the transmembrane glycoprotein CD41 (gpllb), a megakaryocytic marker, is then measured by flow cytometry (see Cwirla, S. E. et al Science, 1997, 276, 1696).

Alpha interferons are a known class of compounds, readily understood by those of skill in the art.

The ability of combinations of thrombopoietin (TPO) receptor agonists, suitably non-peptide TPO receptor agonists, and alpha interferons to treat viral diseases, particularly hepatitis C is demonstrated by activity in the following placebo controlled, double blind, dose-ranging study.

### Study

The safety, tolerability, efficacy and pharmacokinetics of repeat oral daily doses of 3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine);

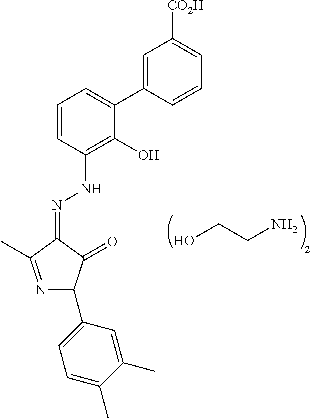

(hereinafter Compound A) in subjects with chronic hepatitis C virus (HCV) associated thrombocytopenia, which precluded initiation of treatment with pegylated interferon (IFN) and ribavirin is assessed in this study.

### Methods

HCV infected subjects with compensated cirrhosis and platelet counts between 20-70,000/ul were randomly allocated to 1 of 4 treatment groups (30, 50, 75 mg eltrombopag or placebo). During Part 1, Compound A was administered in tablet form once daily for 4 weeks. Subjects who achieved platelet counts of >70,000/ul at the end of Part 1 were eligible to enter Part 2, during which subjects initiated therapy with IFN and ribavirin and continued their randomized treatment for a maximum of 12 weeks. A follow-up visit was performed 4 weeks after last dose of study drug. The primary endpoint was defined as an increase in platelet count to 100,000/ul at Week 4.

### Data

| | Placebo | 30 mg | 50 mg | 75 mg |
|---|---|---|---|---|
| | N = 18 | N = 14 | N = 19 | N = 23 |
| Median age (yrs) | 52 | 56 | 50 | 51 |
| Male gender | 11/18 | 10/14 | 12/19 | 19/23 |
| (n/N, %) | (61%) | (71%) | (63%) | (83%) |
| Median platelet count at | 55 | 61 | 53 | 55 |
| Baseline, ×10³/ul | (31, 75) | (34, 94) | (26, 66) | (28, 75) |
| (min, max) | | | | |
| Median platelet count at | 52.5 | 136.5 | 213.5 | 209 |
| Week 4, ×10³/ul | (34, 74) | (40, 528) | (47, 499) | (78, 527) |
| (min, max) | | | | |
| Subjects with platelet | 0/17 | 9/12 | 14/19 | 20/21 |
| count ≧100 × 10³/ul, | (0%) | (75%) | (73.7%) | (95.2%) |
| Week 4 (n/N, %)* | | | | |
| Subjects initiating | 4/18 | 10/14 | 14/19 | 21/23 |
| IFN therapy (n/N, %) | (22%) | (71%) | (74%) | (91%) |
| Subjects completing | 1/18 | 5/14 | 10/19 | 15/23 |
| 12 weeks IFN therapy | (6%) | (36%) | (53%) | (65%) |
| (n/N, %) | | | | |
| Subjects who experienced | 3/18 | 3/14 | 8/19 | 6/23 |
| a drug-related AE at any | (17%) | (21%) | (42%) | (26%) |
| time (n/N, %) | | | | |
| Subjects who WD due to | 0/18 | 2/14 | 1/19 | 1/23 |
| AE at any time (n/N, %) | (0%) | (14%) | (5%) | (4%) |

*Denominator excludes major protocol violators

### Results

The highest response rate at Week 4, with respect to platelets, was observed in the 75 mg Compound A group (20/21, 95%, p<0.0001). Of the 49 subjects successfully initiating IFN therapy at Week 4, 5-15 subjects (36-65%) in the Compound A groups completed 12 weeks of antiviral therapy compared to 1 (6%) subject in the placebo group. Two SAEs were reported on treatment and 4 during follow-up; only one (thrombocytopenia) was considered related to study drug.

### Conclusion

Compound A increased platelet counts in all treatment groups at Day 28 and enabled 71%-91% of subjects to initiate antiviral therapy, with 36-65% of subjects completing 12 weeks of antiviral therapy. These data indicate Compound A in the initiation and maintenance of interferon therapy.

The present invention therefore provides novel combinations of thrombopoietin (TPO) receptor agonists, suitably non-peptide TPO receptor agonists, and antiviral therapy selected from: alpha interferons, including Pegasys® (peginterferon alfa-2a) and Peg-Intron® (peginterferon alfa-2b), ribavirin, ribavirin analogs and HCV therapy, including HCV polymerase inhibitors, helicase inhibitors and protease inhibitors, and the use of these combinations in the treatment of viral diseases, particularly hepatitis C.

The present invention therefore provides novel combinations of thrombopoietin (TPO) receptor agonists, suitably non-peptide TPO receptor agonist, and antiviral therapy selected from: alpha interferons, including Pegasys® (peginterferon alfa-2a) and Peg-Intron® (peginterferon alfa-2b), ribavirin, ribavirin analogs and HCV therapy, including HCV polymerase inhibitors, helicase inhibitors and protease inhibitors, and the use of these combinations in the treatment of viral diseases. Which treatment comprises the co-administration of a therapeutically effective amount of a TPO receptor agonist, suitably a compound of Formula (I), and/or a pharmaceutically acceptable salt thereof, suitably Compound A and an antiviral therapy selected from alpha interferon, particularly Pegasys® (peginterferon alfa-2a) and Peg-Intron® (peginterferon alfa-2b), ribavirin, ribavirin analogs and HCV therapy. The drugs may be administered to a patient in need thereof by any conventional route of administration, including, but not limited to, intravenous, intramuscular, oral, subcutaneous, intradermal, and parenteral.

The TPO receptor agonists, suitably non-peptide TPO receptor agonists, of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The alpha interferons, ribavirin, ribavirin analogs and HCV antivirals of the present invention are incorporated into convenient dosage forms by methods well known to those of skill in the art.

Pegasys® (peginterferon alfa-2a) and Peg-Intron® (peginterferon alfa-2b) are known compounds currently marketed in the United States. Dosage forms of Pegasys® (peginterferon alfa-2a) and Peg-Intron® (peginterferon alfa-2b) for use in the present invention are readily prepared by methods well known to those of skill in the art.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the pharmaceutically active compounds in a pharmaceutical dosage unit as described above will be an efficacious, nontoxic quantity preferably selected from the range of 0.001-100 mg/kg of active compound, preferably 0.002-50 mg/kg. One skilled in the art can readily determine an appropriate dose for treating a human patient with a TPO receptor agonist, according to the present invention. When treating a human patient with non-peptide TPO receptor agonist, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg, more preferably 0.1 to 3000 mg of active compound. Oral administration, which uses lower dosages is preferred. Parenteral administration, at high dosages, however, also can be used when safe and convenient for the patient. One skilled in the art can readily determine an appropriate dose for treating a human patient with an alpha interferon, according to the present invention.

Optimal dosages of the presently invented combinations to be administered may be readily determined by those skilled in the art, and will vary with the particular TPO receptor agonist and antiviral therapy in use, the strength of the preparation, the mode of administration, and the advancement of the disease condition. Additional factors depending on the particular patient being treated will result in a need to adjust dosages, including patient age, weight, diet, and time of administration.

The method of this invention of treating viral diseases, particularly hepatitis C, in mammals, including humans, comprises administering to a subject in need thereof a therapeutically effective amount of a pharmaceutically active combination of the present invention.

The invention also provides for the use of a compound of Formula (I) in the manufacture of a medicament for use in combination with an antiviral therapy selected from: an alpha interferon, ribavirin, a ribavirin analog and an HCV antiviral in the treatment of viral diseases, particularly hepatitis C.

The invention also provides for the use of a compound of Formula (I) in the manufacture of a medicament for use in combination with an alpha interferon for use in therapy.

The invention also provides for a pharmaceutical combination for use in the treatment of viral diseases, particularly hepatitis C, which comprises a compound of Formula (I) and an antiviral therapy selected from: an alpha interferon, ribavirin, a ribavirin analog and an HCV antiviral and a pharmaceutically acceptable carrier.

The invention also provides for the use of Compound A in the manufacture of a medicament for use in combination with an antiviral therapy selected from: an alpha interferon, ribavirin, a ribavirin analog and an HCV antiviral for the treatment of viral diseases, particularly hepatitis C.

The invention also provides for the use of Compound A in the manufacture of a medicament for use in combination with an antiviral therapy selected from: an alpha interferon, ribavirin, a ribavirin analog and an HCV antiviral for use in therapy.

The invention also provides for a combination for use in the treatment of viral diseases, particularly hepatitis C, which comprises Compound A and an antiviral therapy selected from: an alpha interferon, ribavirin, a ribavirin analog and an HCV antiviral and a pharmaceutically acceptable carrier.

No unacceptable toxicological effects are expected when compounds of the invention are administered in accordance with the present invention.

In addition, the combinations of the present invention can be co-administered with further active ingredients, such as other compounds known to treat viral diseases, particularly hepatitis C, or compounds known to have utility when used in combination with a TPO receptor agonist, suitably a non-peptide TPO receptor agonist, or an antiviral therapy selected from: an alpha interferon, ribavirin, a ribavirin analog and an HCV antiviral.

Without further elaboration, it is believed that one skilled in the art can, using the preceding description, utilize the present invention to its fullest extent. The following Examples are, therefore, to be construed as merely illustrative and not a limitation of the scope of the present invention in any way.

### Experimental Details

### EXAMPLE 1

### Capsule Composition

An oral dosage form for administering the present invention is produced by filing a standard two piece hard gelatin capsule with the ingredients in the proportions shown in Table I, below.

TABLE I

| INGREDIENTS | AMOUNTS |
|---|---|
| 3′-{N′-[1-(3,4-Dimethylphenyl)-3-methyl-5-oxo-1,5- | 25 mg |
| dihydropyrazol-4-ylidene]hydrazino}-2′-hydroxybiphenyl-3- | |
| carboxylic acid bis-(monoethanolamine) | |
| Lactose | 55 mg |
| Talc | 16 mg |
| Magnesium Stearate | 4 mg |

### EXAMPLE 2

### Injectable Parenteral Composition

An injectable form for administering the present invention is produced by stirring 1.5% by weight of 3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine) in 10% by volume propylene glycol in water.

### Example 3

### Tablet Composition

The sucrose, calcium sulfate dihydrate and a non-peptide TPO agonist, as shown in Table II below, are mixed and granulated in the proportions shown with a 10% gelatin solution. The wet granules are screened, dried, mixed with the starch, talc and stearic acid, then screened and compressed into a tablet.

TABLE II

| INGREDIENTS | AMOUNTS |
|---|---|
| 3′-{N′-[1-(3,4-Dimethylphenyl)-3-methyl-5-oxo-1,5- | 20 mg |
| dihydropyrazol-4-ylidene]hydrazino}-2′-hydroxybiphenyl- | |
| 3-carboxylic acid bis-(monoethanolamine) | |
| calcium sulfate dihydrate | 30 mg |
| sucrose | 4 mg |
| starch | 2 mg |
| talc | 1 mg |
| stearic acid | 0.5 mg |

While the preferred embodiments of the invention are illustrated by the above, it is to be understood that the invention is not limited to the precise instructions herein disclosed and that the right to all modifications coming within the scope of the following claims is reserved.

1. A method of treating viral diseases in a human in need thereof which comprises the in vivo administration of a TPO receptor agonist and an antiviral therapy selected from: an alpha interferon, ribavirin, a ribavirin analog and an HCV antiviral, and optional further active agents to such human.

2. The method of claim 1 wherein the TPO receptor agonist is a non-peptide TPO receptor agonist and the antiviral therapy is an alpha interferon.

3. (canceled)

4. The method of claim 2 wherein the viral disease is hepatitis C.

5-12. (canceled)

13. The method of claim 4 wherein the non-peptide TPO receptor agonist is 3′-{N′-[1-(3,4-Dimethylphenyl)-3-methyl-5-oxo-1,5-dihydropyrazol-4-ylidene]hydrazino}-2′-hydroxybiphenyl-3-carboxylic acid bis-(monoethanolamine).

14. The method of claim 13 wherein the alpha interferon is peginterferon alfa-2a.

15. The method of claim 13 wherein the alpha interferon is peginterferon alfa-2b.

16. The method of claim 14 further comprising co-administering a therapeutically effective amount of Ribavirin.

17. The method of claim 15 further comprising co-administering a therapeutically effective amount of Ribavirin.

18. The method of claim 1 wherein the TPO receptor agonist is a compound having the following structure: 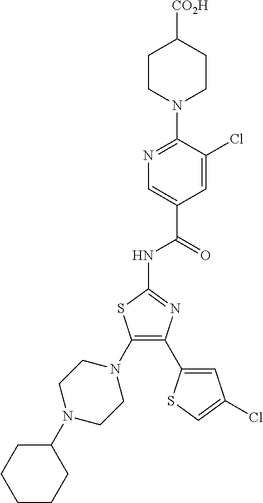
and/or a pharmaceutically acceptable salt thereof.

19. The method of claim 1 wherein the TPO receptor agonist is a compound having the following structure: 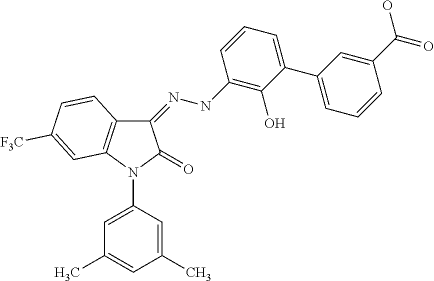
and/or a pharmaceutically acceptable salt thereof.

20. The method of claim 1 wherein the TPO receptor agonist is AMG 531.

21. The method of claim 18 wherein the alpha interferon is peginterferon alfa-2a.

22. The method of claim 18 wherein the alpha interferon is peginterferon alfa-2b.

23. The method of claim 21 further comprising co-administering a therapeutically effective amount of Ribavirin.

24. The method of claim 22 further comprising co-administering a therapeutically effective amount of Ribavirin.

25. The method of claim 19 wherein the alpha interferon is peginterferon alfa-2a.

26. The method of claim 19 wherein the alpha interferon is peginterferon alfa-2b.

29. The method of claim 25 further comprising co-administering a therapeutically effective amount of Ribavirin.

30. The method of claim 26 further comprising co-administering a therapeutically effective amount of Ribavirin.

31. The method of claim 4 wherein the non-peptide TPO receptor agonist is 3′-{N′-[1-(3,4-Dimethylphenyl)-3-methyl-5-oxo-1,5-dihydropyrazol-4-ylidene]hydrazino}-2′-hydroxybiphenyl-3-carboxylic acid,
and/or a pharmaceutically acceptable salt thereof.
